(19) United States
(12) Patent Application Publication
(10) Pub. No.: US 2025/0121089 A1
(43) Pub. Date: Apr. 17, 2025

(54) COMPOSITIONS AND METHODS FOR ENHANCING THE EFFECT OF A TROP-2 SPECIFIC THERAPEUTIC

(71) Applicant: NOVOCURE GMBH, Root D4 (CH)

(72) Inventors: Tali Voloshin-Sela, Haifa (IL); Rita Kelley, Wayne, PA (US)

(21) Appl. No.: 18/913,133

(22) Filed: Oct. 11, 2024

Related U.S. Application Data
(60) Provisional application No. 63/589,757, filed on Oct. 12, 2023.

(51) Int. Cl. A61K 47/68; A61N 1/40; A61P 35/00

(52) U.S. Cl. CPC A61K 47/6851; A61K 47/68037; A61N 1/40; A61P 35/00

(57) ABSTRACT

Disclosed are methods of treating a subject in need thereof comprising applying an alternating electric field, at a frequency for a period of time, to a target site of the subject in need thereof; and administering a Trop-2 specific therapeutic to the subject in need thereof. The disclosed methods can also enhance the efficacy of a Trop-2 specific therapeutic, increase progression-free survival of a subject with cancer, decrease tumor growth and increase killing of cancer cells.

### CROSS-REFERENCE TO RELATED APPLICATIONS

This application claims the benefit of U.S. Provisional Patent Application No. 63/589,757, filed Oct. 12, 2023, each of which is incorporated by reference herein in its entirety.

### BACKGROUND

Due to the ubiquitous expression in cancer cells with a relatively low expression in most normal tissues, Trop-2 represents an excellent candidate as a diagnostic and a therapeutic target, specifically for antibody-based therapy. Nevertheless, relevant toxicities have been reported relating to the role of Trop-2 in healthy tissues

IGF-1 binds Trop-2 leading to modulation of IGF-1 signalling. Trop-2 can also bind the receptor of IGF-1 (IGF-1R), blocking IGF-1 signalling and playing critical roles in cell growth, differentiation, transformation, and metastasis.

Trop-2 overexpression seems to be related to an increased risk of metastasis in patients affected by various cancer types (oral squamous, thyroid, some oesophageal, gastric, colorectal, pancreatic, ovarian, uterine, cervical, prostate, and urinary bladder).

Trop-2's overexpression has also been associated with the increase in tumor growth, proliferation, and metastasis in various epithelial cancers, i.e., head and neck, thyroid, lung, gastrointestinal tract, breast, renal, and gynecological cancers, and glioma

Upregulation of TROP-2 has been found to drive tumor growth while inhibition of TROP2 can prevent cell proliferation and invasion.

### BRIEF SUMMARY

Disclosed are methods of treating a subject in need thereof comprising applying an alternating electric field, at a frequency for a period of time, to a target site of the subject in need thereof; and administering a Trop-2 specific therapeutic to the subject in need thereof.

Disclosed are methods of enhancing efficacy of a Trop-2 specific therapeutic in a subject in need thereof comprising applying an alternating electric field, at a frequency for a period of time, to a target site of the subject in need thereof; and administering a Trop-2 specific therapeutic to the subject in need thereof.

Disclosed are methods of increasing progression-free survival in a subject having cancer comprising applying an alternating electric field, at a frequency for a period of time, to a target site of the subject; and administering a Trop-2 specific therapeutic to the subject.

Disclosed are methods of decreasing tumor growth in a subject in need thereof comprising applying an alternating electric field, at a frequency for a period of time, to a target site of the subject in need thereof; and administering a Trop-2 inhibitor to the subject in need thereof.

Disclosed are methods of killing cancer cells comprising applying alternating electric fields, at a frequency for a period of time, to a population of cells comprising one or more cancer cells; and contacting the population of cells with a Trophoblast cell surface antigen-2 (Trop-2) inhibitor.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a Trop2 signaling pathway.

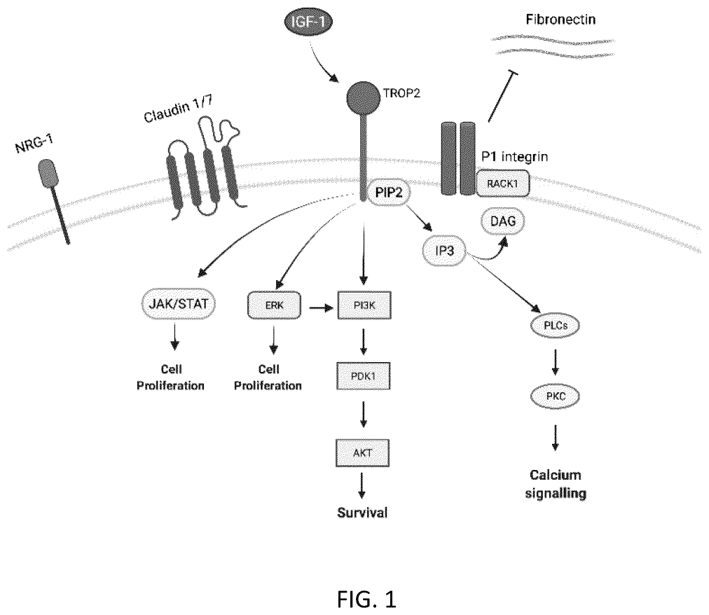

FIG. 2 shows the upregulation of IGF-1 levels following TTFields treatment.

FIG. 3 shows concomitant treatment of TTFields with Sacituzumab Govitecan synergistically inhibited A549 cell growth. Results were analyzed using one-way ANOVA with Tukey's multiple comparisons test. *p<0.05, **p<0.01, ***p<0.001, and ****p<0.0001.

FIG. 4 shows concomitant treatment of TTFields with Sacituzumab Govitecan synergistically elevated the overall effect in A549 cells. Results were analyzed using Student's T test. *p<0.05, **p <0.01, ***p<0.001, and ****p<0.0001.

FIG. 5 shows concomitant treatment of TTFields with Sacituzumab Govitecan enhanced the apoptotic effect in A549 cells. AnnexinV−7AAD—, live cells; AnnexinV+7AAD−, cells at early apoptosis; AnnexinV+7AAD+, cells at late apoptosis.

FIG. 6 shows concomitant treatment of TTFields with Sacituzumab Govitecan synergistically inhibited A2780 cell growth. Results were analyzed using one-way ANOVA with Tukey's multiple comparisons test. *p<0.05, **p<0.01, ***p<0.001, and ****p<0.0001.

FIG. 7 shows concomitant treatment of TTFields with Sacituzumab Govitecan synergistically elevated the overall effect in A2780 cells.

FIG. 8 shows concomitant treatment of TTFields with Sacituzumab Govitecan enhanced the apoptotic effect in A2780 cells. AnnexinV−7AAD−, live cells; AnnexinV+7AAD−, cells at early apoptosis; AnnexinV+7AAD+, cells at late apoptosis.

FIG. 9 shows concomitant treatment of TTFields with Sacituzumab Govitecan synergistically inhibited 4T1 cell growth. Results were analyzed using one-way ANOVA with Tukey's multiple comparisons test. *p<0.05, **p<0.01, ***p<0.001, and ****p<0.0001.

FIG. 10 shows concomitant treatment of TTFields with Sacituzumab Govitecan synergistically elevated the overall effect in 4T1 cells. Results were analyzed using one-way ANOVA with Tukey's multiple comparisons test. *p<0.05, **p<0.01, ***p<0.001, and ****p<0.0001.

FIG. 11 shows concomitant treatment of TTFields with Sacituzumab Govitecan enhanced the apoptotic effect in 4T1 cells.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a Trop-2 specific therapeutic” includes a plurality of such therapeutics, reference to “the cell” is a reference to one or more cells and equivalents thereof known to those skilled in the art, and so forth.

The word “or” as used herein means any one member of a particular list and also includes any combination of members of that list.

As used herein, a “target site” is a specific site or location within or present on a subject or patient. For example, a “target site” can refer to, but is not limited to a cell (e.g., a cancer cell), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, the brain. In some aspects, a cell or population of cells that can be a target site or a target cell include, but are not limited to, a cancer cell (e.g., an ovarian cancer cell). In some aspects, a “target site” can be a tumor target site.

A “tumor target site” is a site or location within or present on a subject or patient that comprises or is adjacent to one or more cancer cells, previously comprised one or more tumor cells, or is suspected of comprising one or more tumor cells. For example, a tumor target site can refer to a site or location within or present on a subject or patient that is prone to metastases. Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g., a target site such as a cell). In some aspects, the alternating electrical field can be in a single direction or multiple directional. In some aspects, alternating electric fields can be delivered through two pairs of transducer arrays that generate perpendicular fields within the target site. For example, for the Optune™ system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and is described in U.S. Pat. No. 7,565,205, which is incorporated herein by reference in its entirety for its teaching of TTFields

In-vivo and in-vitro studies show that the efficacy of TTFields therapy increases as the intensity of the electrical field increases. Therefore, optimizing array placement on a subject to increase the intensity in the target site or target cell is standard practice for the Optune system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g., an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field. For example, the subject of administration can be a subject with ovarian cancer or lung cancer.

By “treat” is meant to administer or apply a therapeutic, such as alternating electric fields and a Trop-2 specific therapeutic, to a subject, such as a human or other mammal (for example, an animal model), that has cancer or has an increased susceptibility for developing cancer, in order to prevent or delay a worsening of the effects of the disease or infection, or to partially or fully reverse the effects of cancer. For example, treating a subject having glioblastoma can comprise delivering a therapeutic to a cell in the subject.

By “prevent” is meant to minimize or decrease the chance that a subject develops cancer.

As used herein, the terms “administering” and “administration” refer to any method of providing a Trop-2 specific therapeutic to a subject directly or indirectly to a target site. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat cancer. In further various aspects, a preparation can be administered prophylactically; that is, administered for prevention of cancer. In an aspect, the skilled person can determine an efficacious dose, an efficacious schedule, or an efficacious route of administration so as to treat a subject. In some aspects, administering comprises contacting, exposing or applying. Thus, in some aspects, exposing a target site or subject to alternating electrical fields or applying alternating electrical fields to a target site or subject or contacting alternating electrical fields to a target site or subject means administering alternating electrical fields to the target site or subject. In some aspects, contacting, exposing and applying can be used interchangeably.

As used herein, “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient”.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise alternating electric fields. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. In some aspects, the frequency of the alternating electric field is between 50 kHz and 1 MHz. The frequency of the alternating electric fields can also be, but is not limited to, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric fields can be electric fields at 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 kHz.

In some aspects, the field strength of the alternating electric fields can be between 0.5 and 4 V/cm RMS. In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some embodiments the field strength is at least 1 V/cm RMS. In some aspects, the field strength can be 0.9 V/cm RMS. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more.

The disclosed methods comprise applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

### C. Trop-2 Specific Therapeutics

In some aspects, a Trop-2 specific therapeutic can be used in combination with an alternating electric field. In some aspects, a Trop-2 specific therapeutic can be, but is not limited to, anti-Trop-2 antibodies and small molecule inhibitors. In some aspects, the anti-Trop-2 antibodies can be conjugated or unconjugated. For example, a conjugated anti-Trop-2 antibody can be conjugated to an anti-cancer therapeutic. In some aspects, examples of anti-Trop-2 antibodies can be, but are not limited to, Sacituzumab Govitecan, Datopotamab Deruxtecan (DS-1062a), PF-06664178, SKB264, JS108, or STI-3258. In some aspects, an unconjugated anti-Trop-2 antibody can be an anti-Trop-2 humanized monoclonal antibody hRS7.

Sacituzumab govitecan is an ADC composed of an anti-Trop-2 humanized monoclonal antibody hRS7 IgG1k coupled with SN-38, an active metabolite of irinotecan (IRI), a topoisomerase I inhibitor. Sacituzumab govitecan is characterized by a high drug-to-antibody ratio (DAR) (7.5-8 molecules of SN-38 conjugated to each antibody via the unique hydrolysable and proprietary linker, CL2A). Sacituzumab govitecan also provides an extracellular release of the drug (payload) that kills surrounding or bystander cells, which do not necessarily express Trop-2 (i.e., bystander killing effect).

Datopotamab is a human IgG1 mAb Trop-2-directed ADC with a potent Topo I inhibitor, deruxtecan (DXd), conjugated through a tetrapeptide-based linker. The tetrapeptide-based linker is enzymatically cleavable and designed to release DXd after proteolytic processing by lysosomal enzymes such as cathepsins.

PF-06664178 is composed of a humanized IgG1 antibody conjugated to microtubule inhibitor AcLys-VCAur0101 linker-payload at the C-terminus of the antibody heavy chain. Upon binding to Trop-2 in the extracellular portion on the cell surface, PF-06664178 is internalized to the lysosomes and processed by proteases to release its auristatin-based Aur0101 payload.

SKB264 is an ADC composed of an anti-Trop-2 humanized monoclonal antibody hRS7 IgG1 conjugated with a proprietary cytotoxic, belotecan-derived payload and stable conjugation chemistry to achieve an average DAR of 7.4. The release of payload upon SKB264 internalization is proportional to the Trop-2 expression. The payload-linker is conjugated to cysteine residues, and the release of the payload relies on hydrolysis, which would happen in the tumor microenvironment, lysosome, and plasma.

In some aspects, an anti-Trop-2 ADC delivers a therapeutic (e.g. cancer therapeutic) to a cell that overexpresses Trop-2 (e.g. cancer cells). In some aspects, an anti-Trop-2 antibody has therapeutic activity without being conjugated.

### D. Compositions

Disclosed are compositions and formulations comprising one or more Trop-2 specific therapeutic with a pharmaceutically acceptable carrier or diluent. In some aspects, the Trop-2 specific therapeutic can be, but is not limited to, Sacituzumab Govitecan, Datopotamab Deruxtecan (DS-1062a), PF-06664178, SKB264, JS108, STI-3258 or a combination thereof. For example, disclosed are pharmaceutical compositions, comprising a Trop-2 specific therapeutic and a pharmaceutically acceptable carrier.

Disclosed are compositions and formulations comprising a Trop-2 specific therapeutic with a pharmaceutically acceptable carrier or diluent. For example, disclosed are pharmaceutical compositions, comprising Sacituzumab govitecan, and a pharmaceutically acceptable carrier or diluent.

In some aspects, the Trop-2 specific therapeutic can be administered with a pharmaceutically acceptable carrier or diluent in any of the disclosed methods.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl choline (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A.R. Gennaro, Mack Publishing Company, Easton, PA 1995, which is hereby incorporated by reference for its teaching of pharmaceutically acceptable carriers. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anaesthetics, and the like. In the methods described herein, delivery of the disclosed compositions to cells can be via a variety of mechanisms. The pharmaceutical composition may be administered in a number of ways depending on whether local or systemic treatment is desired, and on the area to be treated.

### 1. Delivery of Compositions

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid-or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

### E. Methods

### 1. Methods of Treating

Disclosed are methods of treating a subject in need thereof comprising applying an alternating electric field, at a frequency for a period of time, to a target site of the subject in need thereof; and administering a Trop-2 specific therapeutic to the subject in need thereof.

In some aspects, in a subject in need thereof has cancer. In some aspects, the cancer can be, but is not limited to, lung cancer, breast cancer, cervical cancer, colon cancer, gastric cancer, renal cancer, prostate cancer, thyroid cancer, glioblastoma, mesothelioma, pancreatic cancer, non-small cell lung, ovarian cancer, hepatic cancer.

In some aspects, the target site comprises one or more cancer cells. In some aspects, the one or more cancer cells are lung cancer cells, breast cancer cells, cervical cancer cells, colon cancer cells, gastric cancer cells, renal cancer cells, prostate cancer cells, or thyroid cancer cells, glioblastoma cancer cells, mesothelioma cancer cells, pancreatic cancer cells, non-small cell lung cancer cells, ovarian cancer cells, hepatic cancer cells.

In some aspects, one or more cancer cells are killed after treating with an alternating electric field and a Trop-2 specific therapeutic.

In some aspects, the Trop-2 specific therapeutic can be any of those disclosed herein. In some aspects, the Trop-2 specific therapeutic is an antibody-drug conjugate. In some aspects, the antibody-drug conjugate is Sacituzumab Govitecan, Datopotamab Deruxtecan (DS-1062a), PF-06664178, SKB264, JS108, or STI-3258.

In some aspects, the Trop-2 specific therapeutic inhibits IGF-1 signaling. In some aspects, the Trop-2 specific therapeutic blocks IGF-1 from binding to its receptor, IGF-1R.

In some aspects, the alternating electric field is applied before, after, or simultaneously with administering the Trop-2 specific therapeutic. In some aspects, applying an alternating electric field occurs 1, 2, 3, 4, 5, 6, or 7 days prior to administering the Trop-2 specific therapeutic. In some aspects, applying an alternating electric field occurs 1, 2, 3, 4, 5, 6, or 7 days after administering the Trop-2 specific therapeutic. In some aspects, applying alternating electric fields occurs 1, 2, 3, or 4 weeks prior to administering the Trop-2 specific therapeutic. In some aspects, applying alternating electric fields occurs 1, 2, 3, or 4 weeks after administering the Trop-2 specific therapeutic. In some aspects, the alternating electric fields and the Trop-2 specific therapeutic are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the Trop-2 specific therapeutic is present in the bloodstream prior to applying the alternating electric fields.

In some aspects, the Trop-2 specific therapeutic is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

In some aspects, the alternating electric field can have a frequency and field strength. In some aspects, the frequency of the alternating electric field is between 50 and 1 MHz. In some aspects, the frequency of the alternating electric field is 100 kHz-1 MHz. In some aspects, the frequency of the alternating electric field is 100-500 kHz. In some aspects, the frequency of the alternating electric field is 150 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS . . . In some aspects, the alternating electric field has a field strength of 0.9 V/cm RMS. In some aspects, the alternating electric field has a field strength of 1 V/cm RMS. In some aspects, the alternating electric field has a field strength of any of those described herein.

In some aspects, a therapeutically effective amount of a Trop-2 specific therapeutic refers to an amount that is sufficient or effective to prevent or decrease (delay or prevent, inhibit, decrease or reverse) the effects of cancer based on the upregulation of Trop-2. For example, Sacituzumab govitecan, an anti-Trop-2 antibody, can be administered at 10 mg/kg intravenously on Days 1 and 8 of 21-day continuous treatment cycles. In some aspects, a therapeutically effective amount of Datopotamab is 6-8 mg/kg once every 3 weeks. In some aspects, a therapeutically effective amount of PF-06664178 is ranged from 0.15 mg/kg to 4.8 mg/kg intravenously every 21 days. In some aspects, a therapeutically effective amount of SKB264 is 5 mg/kg once every 2 weeks.

### 2. Methods of Enhancing Efficacy

Disclosed are methods of enhancing efficacy of a Trop-2 specific therapeutic in a subject in need thereof comprising applying an alternating electric field, at a frequency for a period of time, to a target site of the subject in need thereof; and administering a Trop-2 specific therapeutic to the subject in need thereof.

In some aspects, in a subject in need thereof has cancer. In some aspects, the cancer can be, but is not limited to, lung cancer, breast cancer, cervical cancer, colon cancer, gastric cancer, renal cancer, prostate cancer, thyroid cancer, glioblastoma, mesothelioma, pancreatic cancer, non-small cell lung, ovarian cancer, hepatic cancer.

In some aspects, the target site comprises one or more cancer cells. In some aspects, the one or more cancer cells are lung cancer cells, breast cancer cells, cervical cancer cells, colon cancer cells, gastric cancer cells, renal cancer cells, prostate cancer cells, or thyroid cancer cells,glioblastoma cancer cells, mesothelioma cancer cells, pancreatic cancer cells, non-small cell lung cancer cells, ovarian cancer cells, hepatic cancer cells.

In some aspects, one or more cancer cells are killed after treating with an alternating electric field and a Trop-2 specific therapeutic.

In some aspects, an increase in efficacy of a Trop-2 specific therapeutic can be compared to a subject receiving a of a Trop-2 specific therapeutic without applying an alternating electric field.

In some aspects, the Trop-2 specific therapeutic is an antibody-drug conjugate. In some aspects, the antibody-drug conjugate is Sacituzumab Govitecan, Datopotamab Deruxtecan (DS-1062a), PF-06664178, SKB264, JS108, or STI-3258.

In some aspects, the Trop-2 specific therapeutic inhibits IGF-1 signaling. In some aspects, the Trop-2 specific therapeutic blocks IGF-1 from binding to its receptor, IGF-1R.

In some aspects, the alternating electric field is applied before, after, or simultaneously with administering the Trop-2 specific therapeutic. In some aspects, applying an alternating electric field occurs 1, 2, 3, 4, 5, 6, or 7 days prior to administering the Trop-2 specific therapeutic. In some aspects, applying an alternating electric field occurs 1, 2, 3, 4, 5, 6, or 7 days after administering the Trop-2 specific therapeutic. In some aspects, applying alternating electric fields occurs 1, 2, 3, or 4 weeks prior to administering the Trop-2 specific therapeutic. In some aspects, applying alternating electric fields occurs 1, 2, 3, or 4 weeks after administering the Trop-2 specific therapeutic. In some aspects, the alternating electric fields and the Trop-2 specific therapeutic are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the Trop-2 specific therapeutic is present in the bloodstream prior to applying the alternating electric fields.

In some aspects, the Trop-2 specific therapeutic is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

In some aspects, the alternating electric field can have a frequency and field strength. In some aspects, the frequency of the alternating electric field is between 50 and 1 MHz. In some aspects, the frequency of the alternating electric field is 100 kHz-1 MHz. In some aspects, the frequency of the alternating electric field is 100-500 kHz. In some aspects, the frequency of the alternating electric field is 150 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS . . . In some aspects, the alternating electric field has a field strength of 0.9 V/cm RMS. In some aspects, the alternating electric field has a field strength of 1 V/cm RMS. In some aspects, the alternating electric field has a field strength of any of those described herein.

In some aspects, a therapeutically effective amount of a Trop-2 specific therapeutic refers to an amount that is sufficient or effective to prevent or decrease (delay or prevent, inhibit, decrease or reverse) the effects of cancer based on the upregulation of Trop-2. For example, Sacituzumab govitecan, an anti-Trop-2 antibody, can be administered at 10 mg/kg intravenously on Days 1 and 8 of 21-day continuous treatment cycles. In some aspects, a therapeutically effective amount of Datopotamab is 6-8 mg/kg once every 3 weeks. In some aspects, a therapeutically effective amount of PF-06664178 is ranged from 0.15 mg/kg to 4.8 mg/kg intravenously every 21 days. In some aspects, a therapeutically effective amount of SKB264 is 5 mg/kg once every 2 weeks.

### 3. Methods of Increasing Progression-Free Survival

Disclosed are methods of increasing progression-free survival in a subject having cancer comprising applying an alternating electric field, at a frequency for a period of time, to a target site of the subject; and administering a Trop-2 specific therapeutic to the subject.

In some aspects, the cancer can be, but is not limited to, lung cancer, breast cancer, cervical cancer, colon cancer, gastric cancer, renal cancer, prostate cancer, thyroid cancer, glioblastoma, mesothelioma, pancreatic cancer, non-small cell lung, ovarian cancer, hepatic cancer.

In some aspects, the target site comprises one or more cancer cells. In some aspects, the one or more cancer cells are lung cancer cells, breast cancer cells, cervical cancer cells, colon cancer cells, gastric cancer cells, renal cancer cells, prostate cancer cells, or thyroid cancer cells, glioblastoma cancer cells, mesothelioma cancer cells, pancreatic cancer cells, non-small cell lung cancer cells, ovarian cancer cells, hepatic cancer cells.

In some aspects, one or more cancer cells are killed after treating with an alternating electric field and a Trop-2 specific therapeutic, thus aiding in the increase in progression-free survival.

In some aspects, the Trop-2 specific therapeutic is an antibody-drug conjugate. In some aspects, the antibody-drug conjugate is Sacituzumab Govitecan, Datopotamab Deruxtecan (DS-1062a), PF-06664178, SKB264, JS108, or STI-3258.

In some aspects, the Trop-2 specific therapeutic inhibits IGF-1 signaling. In some aspects, the Trop-2 specific therapeutic blocks IGF-1 from binding to its receptor, IGF-1R.

In some aspects, the alternating electric field is applied before, after, or simultaneously with administering the Trop-2 specific therapeutic. In some aspects, applying an alternating electric field occurs 1, 2, 3, 4, 5, 6, or 7 days prior to administering the Trop-2 specific therapeutic. In some aspects, applying an alternating electric field occurs 1, 2, 3, 4, 5, 6, or 7 days after administering the Trop-2 specific therapeutic. In some aspects, applying alternating electric fields occurs 1, 2, 3, or 4 weeks prior to administering the Trop-2 specific therapeutic. In some aspects, applying alternating electric fields occurs 1, 2, 3, or 4 weeks after administering the Trop-2 specific therapeutic. In some aspects, the alternating electric fields and the Trop-2 specific therapeutic are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the Trop-2 specific therapeutic is present in the bloodstream prior to applying the alternating electric fields.

In some aspects, the Trop-2 specific therapeutic is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

In some aspects, the alternating electric field can have a frequency and field strength. In some aspects, the frequency of the alternating electric field is between 50 and 1 MHz. In some aspects, the frequency of the alternating electric field is 100 kHz-1 MHz. In some aspects, the frequency of the alternating electric field is 100-500 kHz. In some aspects, the frequency of the alternating electric field is 150 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS . . . In some aspects, the alternating electric field has a field strength of 0.9 V/cm RMS. In some aspects, the alternating electric field has a field strength of 1 V/cm RMS. In some aspects, the alternating electric field has a field strength of any of those described herein.

In some aspects, a therapeutically effective amount of a Trop-2 specific therapeutic refers to an amount that is sufficient or effective to prevent or decrease (delay or prevent, inhibit, decrease or reverse) the effects of cancer based on the upregulation of Trop-2. For example, Sacituzumab govitecan, an anti-Trop-2 antibody, can be administered at 10 mg/kg intravenously on Days 1 and 8 of 21-day continuous treatment cycles. In some aspects, a therapeutically effective amount of Datopotamab is 6-8 mg/kg once every 3 weeks. In some aspects, a therapeutically effective amount of PF-06664178 is ranged from 0.15 mg/kg to 4.8 mg/kg intravenously every 21 days. In some aspects, a therapeutically effective amount of SKB264 is 5 mg/kg once every 2 weeks.

### 4. Methods of Decreasing Tumor Growth

Disclosed are methods of decreasing tumor growth in a subject in need thereof comprising applying an alternating electric field, at a frequency for a period of time, to a target site of the subject in need thereof; and administering a Trop-2 inhibitor to the subject in need thereof.

In some aspects, in a subject in need thereof has cancer. In some aspects, the cancer can be, but is not limited to, lung cancer, breast cancer, cervical cancer, colon cancer, gastric cancer, renal cancer, prostate cancer, thyroid cancer, glioblastoma, mesothelioma, pancreatic cancer, non-small cell lung, ovarian cancer, hepatic cancer.

In some aspects, the target site comprises one or more cancer cells. In some aspects, the one or more cancer cells are lung cancer cells, breast cancer cells, cervical cancer cells, colon cancer cells, gastric cancer cells, renal cancer cells, prostate cancer cells, or thyroid cancer cells,glioblastoma cancer cells, mesothelioma cancer cells, pancreatic cancer cells, non-small cell lung cancer cells, ovarian cancer cells, hepatic cancer cells.

In some aspects, one or more cancer cells are killed after treating with an alternating electric field and a Trop-2 specific therapeutic, thus decreasing tumor growth.

In some aspects, the Trop-2 specific therapeutic is an antibody-drug conjugate. In some aspects, the antibody-drug conjugate is Sacituzumab Govitecan, Datopotamab Deruxtecan (DS-1062a), PF-06664178, SKB264, JS108, or STI-3258.

In some aspects, the Trop-2 specific therapeutic inhibits IGF-1 signaling. In some aspects, the Trop-2 specific therapeutic blocks IGF-1 from binding to its receptor, IGF-1R.

In some aspects, the alternating electric field is applied before, after, or simultaneously with administering the Trop-2 specific therapeutic. In some aspects, applying an alternating electric field occurs 1, 2, 3, 4, 5, 6, or 7 days prior to administering the Trop-2 specific therapeutic. In some aspects, applying an alternating electric field occurs 1, 2, 3, 4, 5, 6, or 7 days after administering the Trop-2 specific therapeutic. In some aspects, applying alternating electric fields occurs 1, 2, 3, or 4 weeks prior to administering the Trop-2 specific therapeutic. In some aspects, applying alternating electric fields occurs 1, 2, 3, or 4 weeks after administering the Trop-2 specific therapeutic. In some aspects, the alternating electric fields and the Trop-2 specific therapeutic are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the Trop-2 specific therapeutic is present in the bloodstream prior to applying the alternating electric fields.

In some aspects, the Trop-2 specific therapeutic is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

In some aspects, the alternating electric field can have a frequency and field strength. In some aspects, the frequency of the alternating electric field is between 50 and 1 MHz. In some aspects, the frequency of the alternating electric field is 100 kHz-1 MHz. In some aspects, the frequency of the alternating electric field is 100-500 kHz. In some aspects, the frequency of the alternating electric field is 150 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS . . . In some aspects, the alternating electric field has a field strength of 0.9 V/cm RMS. In some aspects, the alternating electric field has a field strength of 1 V/cm RMS. In some aspects, the alternating electric field has a field strength of any of those described herein.

In some aspects, a therapeutically effective amount of a Trop-2 specific therapeutic refers to an amount that is sufficient or effective to prevent or decrease (delay or prevent, inhibit, decrease or reverse) the effects of cancer based on the upregulation of Trop-2. For example, Sacituzumab govitecan, an anti-Trop-2 antibody, can be administered at 10 mg/kg intravenously on Days 1 and 8 of 21-day continuous treatment cycles. In some aspects, a therapeutically effective amount of Datopotamab is 6-8 mg/kg once every 3 weeks. In some aspects, a therapeutically effective amount of PF-06664178 is ranged from 0.15 mg/kg to 4.8 mg/kg intravenously every 21 days. In some aspects, a therapeutically effective amount of SKB264 is 5 mg/kg once every 2 weeks.

### 5. Methods of Killing

Disclosed are methods of killing cancer cells comprising applying alternating electric fields, at a frequency for a period of time, to a population of cells comprising one or more cancer cells; and contacting the population of cells with a Trophoblast cell surface antigen-2 (Trop-2) inhibitor.

In some aspects, the one or more cancer cells are lung cancer cells, breast cancer cells, cervical cancer cells, colon cancer cells, gastric cancer cells, renal cancer cells, prostate cancer cells, or thyroid cancer cells,glioblastoma cancer cells, mesothelioma cancer cells, pancreatic cancer cells, non-small cell lung cancer cells, ovarian cancer cells, hepatic cancer cells.

In some aspects, the population of cells is in vitro. In some aspects, the population of cells is in a subject.

In some aspects, the Trop-2 specific therapeutic is an antibody-drug conjugate. In some aspects, the antibody-drug conjugate is Sacituzumab Govitecan, Datopotamab Deruxtecan (DS-1062a), PF-06664178, SKB264, JS108, or STI-3258.

In some aspects, the Trop-2 specific therapeutic inhibits IGF-1 signaling. In some aspects, the Trop-2 specific therapeutic blocks IGF-1 from binding to its receptor, IGF-1R.

In some aspects, the alternating electric field is applied before, after, or simultaneously with administering the Trop-2 specific therapeutic. In some aspects, applying an alternating electric field occurs 1, 2, 3, 4, 5, 6, or 7 days prior to administering the Trop-2 specific therapeutic. In some aspects, applying an alternating electric field occurs 1, 2, 3, 4, 5, 6, or 7 days after administering the Trop-2 specific therapeutic. In some aspects, applying alternating electric fields occurs 1, 2, 3, or 4 weeks prior to administering the Trop-2 specific therapeutic. In some aspects, applying alternating electric fields occurs 1, 2, 3, or 4 weeks after administering the Trop-2 specific therapeutic. In some aspects, the alternating electric fields and the Trop-2 specific therapeutic are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the Trop-2 specific therapeutic is present in the bloodstream prior to applying the alternating electric fields.

In some aspects, the Trop-2 specific therapeutic is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

In some aspects, the alternating electric field can have a frequency and field strength. In some aspects, the frequency of the alternating electric field is between 50 and 1 MHz. In some aspects, the frequency of the alternating electric field is 100 kHz-1 MHz. In some aspects, the frequency of the alternating electric field is 100-500 kHz. In some aspects, the frequency of the alternating electric field is 150 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS . . . In some aspects, the alternating electric field has a field strength of 0.9 V/cm RMS. In some aspects, the alternating electric field has a field strength of 1 V/cm RMS. In some aspects, the alternating electric field has a field strength of any of those described herein.

In some aspects, a therapeutically effective amount of a Trop-2 specific therapeutic refers to an amount that is sufficient or effective to prevent or decrease (delay or prevent, inhibit, decrease or reverse) the effects of cancer based on the upregulation of Trop-2. For example, Sacituzumab govitecan, an anti-Trop-2 antibody, can be administered at 10 mg/kg intravenously on Days 1 and 8 of 21-day continuous treatment cycles. In some aspects, a therapeutically effective amount of Datopotamab is 6-8 mg/kg once every 3 weeks. In some aspects, a therapeutically effective amount of PF-06664178 is ranged from 0.15 mg/kg to 4.8 mg/kg intravenously every 21 days. In some aspects, a therapeutically effective amount of SKB264 is 5 mg/kg once every 2 weeks.

### F. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of Trop-2 specific therapeutics and one or more materials for delivering alternating electric fields, such as the Optune system. For example disclosed are kits comprising one or more of Sacituzumab Govitecan, Datopotamab Deruxtecan (DS-1062a), PF-06664178, SKB264 JS108, or STI-3258 and one or more materials for delivering alternating electric fields, such as the Optune system.

### G. Illustrative Embodiments

Embodiment 1 describes a method of treating a subject in need thereof comprising: applying an alternating electric field, at a frequency for a period of time, to a target site of the subject in need thereof; and administering a Trophoblast cell surface antigen-2 (Trop-2) specific therapeutic to the subject in need thereof.

Embodiment 2 describes method of enhancing efficacy of a Trophoblast cell surface antigen-2 (Trop-2) inhibitor in a subject in need thereof comprising applying an alternating electric field, at a frequency for a period of time, to a target site of the subject in need thereof; and administering a Trop-2 specific therapeutic to the subject in need thereof.

Embodiment 3 describes embodiments 1-2, wherein a subject in need thereof has cancer.

Embodiment 4 describes method of increasing progression-free survival in a subject having cancer comprising applying an alternating electric field, at a frequency for a period of time, to a target site of the subject; and administering a Trop-2 specific therapeutic to the subject.

Embodiment 5 describes a method of decreasing tumor growth in a subject in need thereof comprising applying an alternating electric field, at a frequency for a period of time, to a target site of the subject in need thereof; and administering a Trop-2 specific therapeutic to the subject in need thereof.

Embodiment 6 describes embodiments 3-5, wherein the cancer is lung cancer, breast cancer, cervical cancer, colon cancer, gastric cancer, renal cancer, prostate cancer, thyroid cancer, glioblastoma, mesothelioma, pancreatic cancer, non-small cell lung, ovarian cancer, hepatic cancer.

Embodiment 7 describes embodiments 1-6, wherein the target site comprises one or more cancer cells.

Embodiment 8 describes embodiment 7, wherein the one or more cancer cells are lung cancer cells, breast cancer cells, cervical cancer cells, colon cancer cells, gastric cancer cells, renal cancer cells, prostate cancer cells, or thyroid cancer cells,glioblastoma cancer cells, mesothelioma cancer cells, pancreatic cancer cells, non-small cell lung cancer cells, ovarian cancer cells, hepatic cancer cells.

Embodiment 9 describes embodiments 7-8, wherein one or more cancer cells are killed.

Embodiment 10 describes a method of killing cancer cells comprising applying alternating electric fields, at a frequency for a period of time, to a population of cells comprising one or more cancer cells; and contacting the population of cells with a Trophoblast cell surface antigen-2 (Trop-2) specific therapeutic.

Embodiment 11 describes embodiment 10, wherein the population of cells is in vitro.

Embodiment 12 describes embodiment 10, wherein the population of cells is in a subject.

Embodiment 13 describes embodiments 1-12, wherein the Trop-2 specific therapeutic is an antibody-drug conjugate.

Embodiment 14 describes embodiment 13, wherein the antibody-drug conjugate is sacituzumab govitecan, PF-06664178, datopotamab deruxtecan, JS108, SKB264, or STI-3258.

Embodiment 15 describes embodiments 1-14, wherein the alternating electric field is applied before, after, or simultaneously with administering the Trop-2 specific therapeutic.

Embodiment 16 describes embodiments 1-15, wherein the Trop-2 specific therapeutic is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

Embodiment 17 describes embodiments 1-16, wherein the TROP-2 specific therapeutic inhibits IGF-1 signaling.

Embodiment 18 describes embodiments 1-17, wherein the Trop-2 specific therapeutic blocks IGF-1 from binding to its receptor, IGF-1R.

Embodiment 19 describes embodiments 1-18, wherein the frequency of the alternating electric field is between 50 kHz and 1 MHz.

Embodiment 20 describes embodiments 1-19, wherein the frequency of the alternating electric field is about 150 or 200 kHz.

Embodiment 21 describes embodiments 1-20, wherein the alternating electric field has a field strength of between 0.5 and 10 V/cm RMS.

Embodiment 22 describes embodiments 1-21, wherein the alternating electric field has a field strength of about 0.9 V/cm RMS.

Embodiment 23 describes embodiments 1-22, wherein administering a Trop-2 specific therapeutic is performed 1, 2, 3, 4, 5, 6, 7, 8, 9, or 10 days after applying an alternating electric field is performed.

Embodiment 24 describes a combination of alternating electric fields and a Trop-2 specific therapeutic for use in treating a subject in need thereof.

Embodiment 25 describes a combination of alternating electric fields and a Trop-2 specific therapeutic for use in enhancing efficacy of a Trop-2 specific therapeutic in a subject in need thereof.

Embodiment 26 describes embodiments 24-25, wherein a subject in need thereof has cancer.

Embodiment 27 describes a combination of alternating electric fields and a Trop-2 specific therapeutic for use in increasing progression-free survival in a subject having cancer.

Embodiment 28 describes a combination of alternating electric fields and a Trop-2 specific therapeutic for use in decreasing tumor growth in a subject in need thereof.

Embodiment 29 describes embodiments 24-28, wherein the cancer is lung cancer, breast cancer, cervical cancer, colon cancer, gastric cancer, renal cancer, prostate cancer, thyroid cancer, glioblastoma, mesothelioma, pancreatic cancer, non-small cell lung, ovarian cancer, hepatic cancer.

Embodiment 30 describes embodiments 24-29, wherein the target site comprises one or more cancer cells.

Embodiment 31 describes embodiment 30, wherein the one or more cancer cells are lung cancer cells, breast cancer cells, cervical cancer cells, colon cancer cells, gastric cancer cells, renal cancer cells, prostate cancer cells, or thyroid cancer cells, glioblastoma cancer cells, mesothelioma cancer cells, pancreatic cancer cells, non-small cell lung cancer cells, ovarian cancer cells, hepatic cancer cells.

Embodiment 32 describes embodiments 24-31, wherein one or more cancer cells are killed.

Embodiment 33 describes a combination of alternating electric fields and a Trop-2 specific therapeutic for use in killing cancer cells.

Embodiment 34 describes embodiment 33, wherein the population of cells is in vitro.

Embodiment 35 describes embodiment 33, wherein the population of cells is in a subject.

Embodiment 36 describes embodiments 24-35, wherein the Trop-2 specific therapeutic is an antibody-drug conjugate.

Embodiment 37 describes embodiment 36, wherein the antibody-drug conjugate is sacituzumab govitecan, PF-06664178, datopotamab deruxtecan, JS108, SKB264, or STI-3258.

Embodiment 38 describes embodiments 24-37, wherein the alternating electric fieldis applied before, after, or simultaneously with administering the Trop-2 specific therapeutic.

Embodiment 39 describes embodiments 24-38, wherein the Trop-2 specific therapeutic is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

Embodiment 40 describes embodiments 24-39, wherein the TROP-2 specific therapeutic inhibits IGF-1 signaling.

Embodiment 41 describes embodiments 24-40, wherein the Trop-2 specific therapeutic blocks IGF-1 from binding to its receptor, IGF-1R.

Embodiment 42 describes embodiments 24-41, wherein the frequency of the alternating electric field is between 50 kHz and 1 MHz.

Embodiment 43 describes embodiments 24-42, wherein the frequency of the alternating electric field is about 150 or 200 kHz.

Embodiment 44 describes embodiments 24-43, wherein the alternating electric field has a field strength of between 0.5 and 10 V/cm RMS.

Embodiment 45 describes embodiments 24-44, wherein the alternating electric field has a field strength of about 0.9 V/cm RMS.

Embodiment 46 describes embodiments 24-45, wherein administering a Trop-2 specific therapeutic is performed 1, 2, 3, 4, 5, 6, 7, 8, 9, or 10 days after applying an alternating electric field is performed.

Embodiment 47 describes a kit comprising one or more Trop-2 specific therapeutics and one or more materials for delivering an alternating electric field.

### EXAMPLES

Trop-2 is a transmembrane glycoprotein that is upregulated in all cancer types independent of baseline levels of Trop-2 expression. Trop-2 is an ideal candidate for targeted therapeutics due to it being a transmembrane protein with an extracellular domain overexpressed on a wide variety of tumors as well as its upregulated expression relative to normal cells [5]. As a result, several Trop-2-targeted therapeutics have recently been developed for clinical use, such as anti-Trop-2 antibodies and Trop-2-targeted antibody—drug conjugates (ADC).

Trop 2 has been reported to bind to several proteins, such as insulin-like growth factor 1 (IGF-1), claudin-1 and -7, cyclin D1, and PKC, and be involved in various cellular processes, including proliferation and Apoptosis.

Furthermore, Trop-2 is a strong predictor of cancer response to AKT inhibitors, and as such AKT inhibitors seem to be a rational target for further investigation along with Trop-2-targeted therapy.

Since TTFields strongly activate Akt signaling it is highly relevant to examine TROP-2 activation following TTFields application.

Subsequently, multiple early-phase clinical trials have demonstrated safety and clinical benefit of Trop-2-based ADCs across multiple tumor types. This includes clinical benefit and tolerability in tumor types with limited treatment options, such as triple-negative breast cancer, platinum-resistant urothelial cancer, and small-cell lung cancer [1].

### A. Example 1

Conditioned media of A2780 ovarian cancer cell line following 1 week of TTFields application can be taken and assessed by Elisa for IGF-1 levels compared with conditioned media of cells that had been grown for one week without TTFields application.

Serums derived from mice baring lung/ovarian tumors and treated with TTFields for at least 7 days can be examined by Elisa for circulating IGF-1 levels compared with serum derived from heat treated mice.

If these experiments show significant differences, secretion of IGF-1 following TTFields application can be studied in additional cell lines and serums.

TROP-2 levels of expression in tissue and cells can be examined by IHC, western blot and RT-PCR.

Inhibition of TROP-2 with an inhibitor can also be examined concomitant to TTFields in NSCLC and Breast cancer cell lines.

### B. Example 2

Tests were performed on three different cell types: A549, A2780 and 4T1. A549 cells are adenocarcinomec human alveolar basal epithelial cells. A2780 is a human ovarian cancer cell line. 4T1 is a breast cancer cell line derived from the mammary gland tissue of a mouse BALB/c strain.

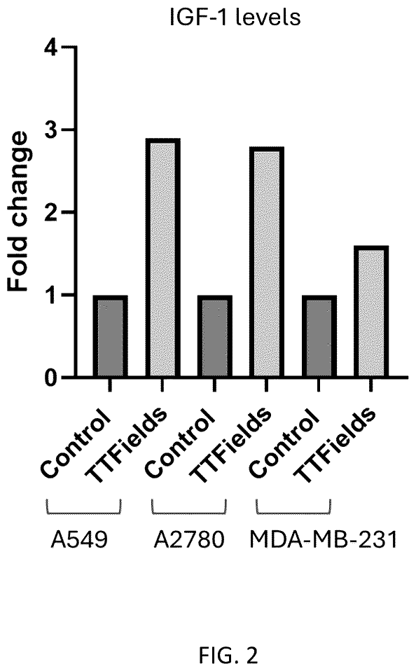

FIG. 2 shows the upregulation of IGF-1 levels following TTFields treatment. Conditioned media (CM) was collected from A549 Non-small-cell lung cancer (NSCLC), A2780 (ovarian carcinoma) and MDA-MB-231 (triple negative breast adenocarcinoma) cells following 72 h of TTFields application. IGFF-1 levels were assessed using IGF-1 ELISA and compared with CM collected from cells grown for 72 h without TTFields application. TTFields upregulated IGF-1 levels in all three cell lines.

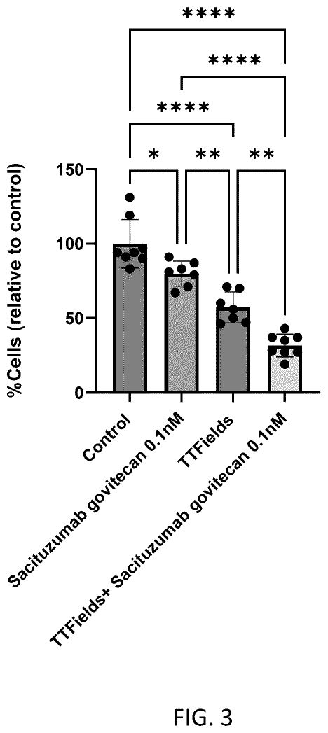

NSCLC cells A549 were treated for 72 h with TTFields at an intensity of 1.62 V/cm RMS and a frequency of 150 kHz using the Inovitro system. TTFields were applied to the cells in the absence or presence of the Trop2 inhibitor Sacituzumab Govitecan. Treated cells were counted using a flow cytometer, and cytotoxic effect was calculated relative to control cells. Results are shown in FIG. 3

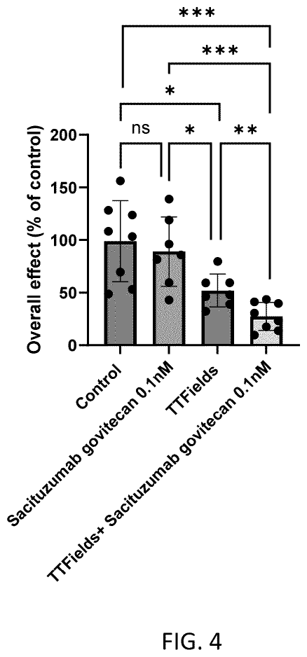

Concomitant treatment of TTFields with Sacituzumab Govitecan synergistically elevated the overall effect in A549 cells (FIG. 4). NSCLC cells A549 were treated for 72 h with TTFields at an intensity of 1.62 V/cm RMS and a frequency of 150 kHz using the Inovitro system. TTFields were applied to the cells in the absence or presence of the Trop2 inhibitor Sacituzumab Govitecan. Treated cells were harvested, re-plated, and grown for an additional 7-14 days. Colonies were stained (0.5% crystal violet solution), counted, and clonogenic effect was calculated relative to control. Overall effect was calculated by multiplying the cytotoxic and clonogenic effects.

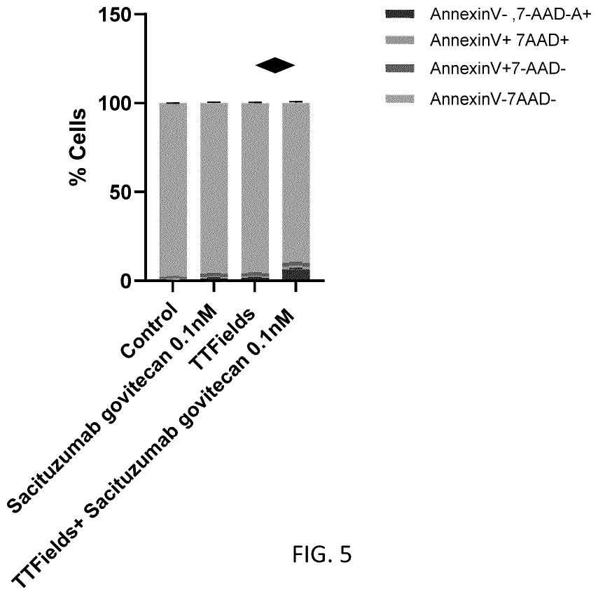

FIG. 5 shows concomitant treatment of TTFields with Sacituzumab Govitecan enhanced the apoptotic effect in A549 cells. NSCLC cells A549 were treated for 72 h with TTFields at an intensity of 1.62 V/cm RMS and a frequency of 150 kHz using the Inovitro system. TTFields were applied to the cells in the absence or presence of the Trop2 inhibitor Sacituzumab Govitecan. Treated cells were double-stained with FITC-conjugated annexin V and 7-aminoactinomycin D (7AAD), and data acquisition was performed using a flow cytometer.

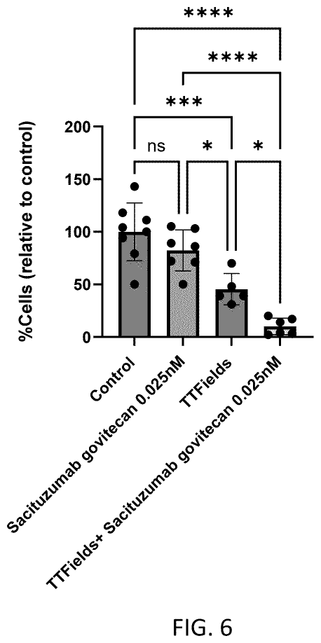

Concomitant treatment of TTFields with Sacituzumab Govitecan synergistically inhibited A2780 cell growth (FIG. 6). Ovarian carcinoma cells A2780 were treated for 72 h with TTFields at an intensity of 1.62 V/cm RMS and a frequency of 200 kHz using the Inovitro system. TTFields were applied to the cells in the absence or presence of the Trop2 inhibitor Sacituzumab Govitecan. Treated cells were counted using a flow cytometer, and cytotoxic effect was calculated relative to control cells.

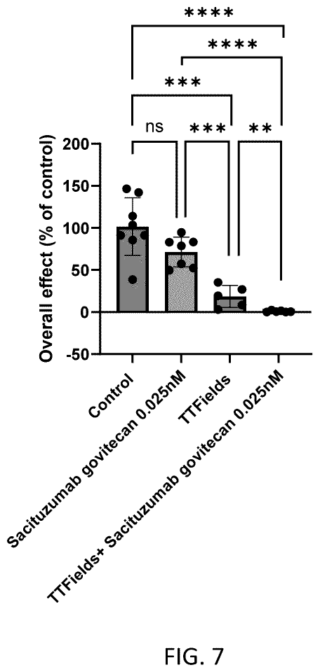

Similar to A549 cells shown in FIG. 4, concomitant treatment of TTFields with Sacituzumab Govitecan synergistically elevated the overall effect in A2780 cells (FIG. 7). Ovarian carcinoma cells A2780 were treated for 72 h with TTFields at an intensity of 1.62 V/cm RMS and a frequency of 200 kHz using the Inovitro system. TTFields were applied to the cells in the absence or presence of the Trop2 inhibitor Sacituzumab Govitecan. Treated cells were harvested, re-plated, and grown for an additional 7-14 days. Colonies were stained (0.5% crystal violet solution), counted, and clonogenic effect was calculated relative to control. Overall effect was calculated by multiplying the cytotoxic and clonogenic effects.

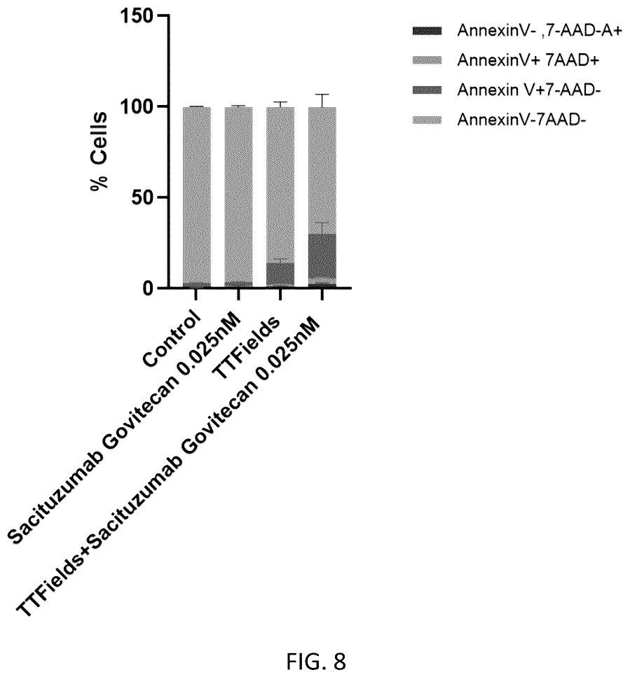

FIG. 8 shows concomitant treatment of TTFields with Sacituzumab Govitecan enhanced the apoptotic effect in A2780 cells. Ovarian carcinoma cells A2780 were treated for 72 h with TTFields at an intensity of 1.62 V/cm RMS and a frequency of 200 kHz using the Inovitro system. TTFields were applied to the cells in the absence or presence of the Trop2 inhibitor Sacituzumab Govitecan. Treated cells were double-stained with FITC-conjugated annexin V and 7-aminoactinomycin D (7AAD), and data acquisition was performed using a flow cytometer.

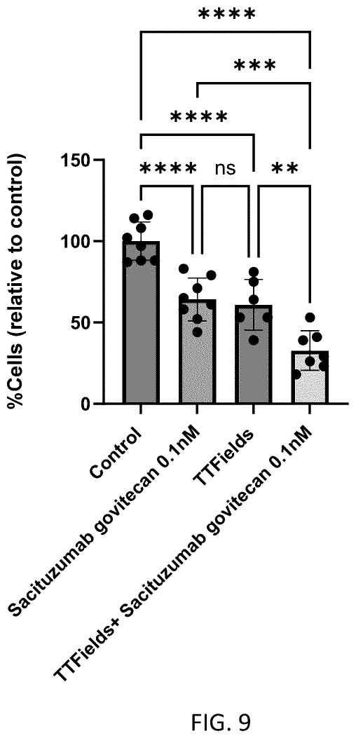

Similar to A549 cells (FIGS. 3) and A2780 cells (FIG. 6), concomitant treatment of TTFields with Sacituzumab Govitecan synergistically inhibited 4T1 cell growth (FIG. 9). Mammary carcinoma cells 4T1 were treated for 72 h with TTFields at an intensity of 1.62 V/cm RMS and a frequency of 150 kHz using the Inovitro system. TTFields were applied to the cells in the absence or presence of the Trop2 inhibitor Sacituzumab Govitecan. Treated cells were counted using a flow cytometer, and cytotoxic effect was calculated relative to control cells.

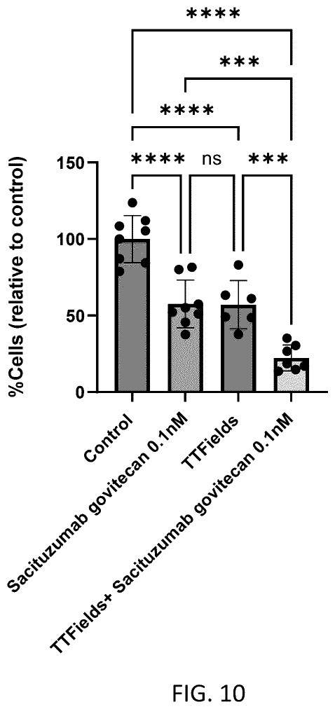

Similar to A549 cells (FIGS. 4) and A2780 cells (FIG. 7), concomitant treatment of TTFields with Sacituzumab Govitecan synergistically elevated the overall effect in 4T1 cells (FIG. 10). Mammary carcinoma cells 4T1 were treated for 72 h with TTFields at an intensity of 1.62 V/cm RMS and a frequency of 150 kHz using the Inovitro system. TTFields were applied to the cells in the absence or presence of the Trop2 inhibitor Sacituzumab Govitecan. Treated cells were harvested, re-plated, and grown for an additional 7-14 days. Colonies were stained (0.5% crystal violet solution), counted, and clonogenic effect was calculated relative to control. Overall effect was calculated by multiplying the cytotoxic and clonogenic effects.

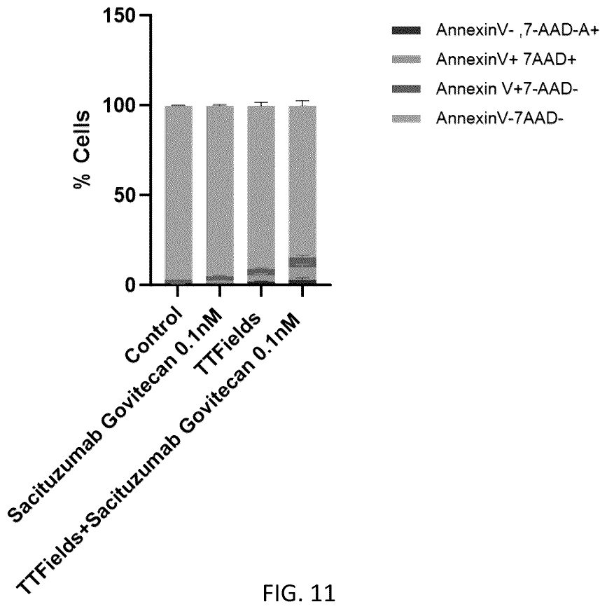

Lastly, like A549 cells (FIGS. 5) and A2780 cells (FIG. 8), concomitant treatment of TTFields with Sacituzumab Govitecan enhanced the apoptotic effect in 4T1 cells (FIG. 11). Mammary carcinoma cells 4T1 were treated for 72 h with TTFields at an intensity of 1.62 V/cm RMS and a frequency of 150 kHz using the Inovitro system. TTFields were applied to the cells in the absence or presence of the Trop2 inhibitor Sacituzumab Govitecan. Treated cells were double-stained with FITC-conjugated annexin V and 7-aminoactinomycin D (7AAD), and data acquisition was performed using a flow cytometer. AnnexinV−7AAD−, live cells; AnnexinV+7AAD−, cells at early apoptosis; AnnexinV+7AAD+, cells at late apoptosis.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of treating a subject in need thereof comprising:
a) applying an alternating electric field, at a frequency for a period of time, to a target site of the subject in need thereof; and
b) administering a Trophoblast cell surface antigen-2 (Trop-2) specific therapeutic to the subject in need thereof.

2. A method of enhancing efficacy of a Trophoblast cell surface antigen-2 (Trop-2) inhibitor in a subject in need thereof comprising:
a) applying an alternating electric field, at a frequency for a period of time, to a target site of the subject in need thereof; and
b) administering a Trop-2 specific therapeutic to the subject in need thereof.

3. The method of claim 1, wherein a subject in need thereof has cancer.

4. The method of claim 3, wherein the cancer is lung cancer, breast cancer, cervical cancer, colon cancer, gastric cancer, renal cancer, prostate cancer, thyroid cancer, glioblastoma, mesothelioma, pancreatic cancer, non-small cell lung, ovarian cancer, hepatic cancer.

5. The method of claim 1, wherein the target site comprises one or more cancer cells.

6. The method of claim 5, wherein the one or more cancer cells are lung cancer cells, breast cancer cells, cervical cancer cells, colon cancer cells, gastric cancer cells, renal cancer cells, prostate cancer cells, or thyroid cancer cells, glioblastoma cancer cells, mesothelioma cancer cells, pancreatic cancer cells, non-small cell lung cancer cells, ovarian cancer cells, hepatic cancer cells.

7. The method of claim 6, wherein one or more cancer cells are killed.

8. A method of killing cancer cells comprising:
a) applying alternating electric fields, at a frequency for a period of time, to a population of cells comprising one or more cancer cells; and
b) contacting the population of cells with a Trophoblast cell surface antigen-2 (Trop-2) specific therapeutic.

9. The method of claim 8, wherein the population of cells is in vitro.

10. The method of claim 8, wherein the population of cells is in a subject.

11. The method of claim 1, wherein the Trop-2 specific therapeutic is an antibody-drug conjugate.

12. The method of claim 11, wherein the antibody-drug conjugate is sacituzumab govitecan, PF-06664178, datopotamab deruxtecan, JS108, SKB264, or STI-3258.

13. The method of claim 1, wherein the alternating electric field is applied before, after, or simultaneously with administering the Trop-2 specific therapeutic.

14. The method of claim 1, wherein the Trop-2 specific therapeutic is administered intravenously, intratumorally, intracranially, intraventricularly, intrathecally, epidurally, intradurally, intravascularly, intraarterially, intramuscularly, subcutaneously, intraperitoneally, orally, intranasally, topically, via intratumor injection, or via inhalation.

15. The method of claim 1, wherein the TROP-2 specific therapeutic inhibits IGF-1 signaling.

16. The method of claim 1, wherein the Trop-2 specific therapeutic blocks IGF-1 from binding to its receptor, IGF-1R.

17. The method of claim 1, wherein the frequency of the alternating electric field is between 50 kHz and 1 MHz.

18. The method of claim 1, wherein the frequency of the alternating electric field is about 150 or 200 kHz.

19. The method of claim 1, wherein the alternating electric field has a field strength of between 0.5 and 10 V/cm RMS.

20. The method of claim 1, wherein step b) is performed 1, 2, 3, 4, 5, 6, 7, 8, 9, or 10 days after step a) is performed.
